(19) United States
(12) Patent Application Publication
(10) Pub. No.: US 2007/0036857 A1
(43) Pub. Date: Feb. 15, 2007

(54) Pharmaceutical multiparticulate composition comprising mycophenolic acid or mycophenolate sodium and combination with rapamycin

(76) Inventors: Dieter Becker, Freiburg (DE); Carsten Burger, Lorrach (DE); Gilles Feutren, Mulhouse (FR); Patrice Guitard, Hegenheim (FR); Andrea Kramer, Reute (DE); Nicoletta Loggia, Basel (CH); Christian-Peter Luftensteiner, Basel (CH); Jorg Ogorka, Steinbrunnen (DE); Harald Ottinger, Freiburg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/570,752

(22) PCT Filed: Oct. 1, 2004
(86) PCT No.: PCT/EP04/10998

§ 371 Date: Aug. 17, 2006

(30) Foreign Application Priority Data
Oct. 3, 2003 (GB) 0323202.2
Oct. 8, 2003 (GB) 0323598.3
Dec. 23, 2003 (GB) 0329852.8
Mar. 16, 2004 (GB) 0405902.8
May 13, 2004 (GB) 0410714.0
Aug. 31, 2004 (GB) 0419356.1

(51) Int. Cl. A61K 31/365; A61K 9/26; A61K 9/14

(52) U.S. Cl. 424/470; 514/469; 424/489

(57) ABSTRACT

The present invention relates to a novel composition of mycophenolic acid, a salt or a prodrug thereof and to a fixed combination of mycophenolic acid, a salt or a prodrug thereof and rapamycin or a rapamycin derivative.

The present invention relates to a novel composition of mycophenolic acid, a salt or a prodrug thereof and to a fixed combination of mycophenolic acid, a salt or a prodrug thereof and rapamycin or a rapamycin derivative.

Mycophenolic acid, also referred to herein as MPA, was first isolated in 1896, and is known to have e.g. anti-tumor, anti-viral, immunosuppressive, anti-psoriatic, and anti-inflammatory activity.

Suitable MPA salts include cationic salts, e.g. alkali metal salts, especially the sodium salt, e.g. mono or di-sodium salt, preferably mono-sodium salt.

Prodrugs of MPA include e.g. physiologically hydrolysable esters of MPA, e.g. as disclosed in U.S. Pat. No. 4,753,935 such as the morpholinoethyl ester, also known as mycophenolate mofetil (MMF).

Preferred is sodium mycophenolate salt.

Mycophenolate salts when enteric coated or adapted to be released in the upper part of the intestines lead to effective, well-tolerated, pharmaceuticals particularly for immuno-suppressive indications, e.g. treatment or prevention of cell, tissue or organ allograft rejection. However, there is still a need to reduce inter- and intrapatient variability, e.g. to reduce variability of drug exposure in the body or food effect, or to further reduce GI side-effects.

Accordingly, the present invention provides:
1. A composition comprising MPA, a salt or a prodrug thereof, e.g. MMF, in a multiparticulate form, e.g. microparticles, minitablets, pellets, granules or beads. Preferably, the composition is enteric coated.

According to a further embodiment of the invention, there is provided:
2. Use of a composition comprising as active ingredient MPA, a salt or a prodrug thereof, e.g. MMF, in a particulate form, e.g. microparticles, minitablets, pellets, granules or beads, to reduce inter- and intrapatient variability, e.g. to reduce variability of drug exposure and/or to reduce or prevent food effect and/or to reduce GI side-effects in a subject.
3. A method for reducing inter- and intrapatient variability, e.g. reducing variability of drug exposure and/or reducing or preventing food effect and/or reduce the GI effects in a subject, e.g. a transplanted subject or a subject having an autoimmune disease, comprising administering a therapeutically effective amount of a composition comprising as active ingredient MPA, a salt or a prodrug thereof, e.g. MMF, wherein the composition is in a particulate form, e.g. microparticles, minitablets, pellets, granules or beads.

The composition of the invention may also be in the form of a tablet or minitablet which disintegrates and/or in the form of a capsule which dissolves, e.g. in the mouth, stomach or small intestine, to give multiparticles, e.g. enteric coated microparticles, pellets or granules. Preferably the composition of the invention is in a particulate form.

Preferably the composition of the invention is enteric coated. By enteric coated or coating is meant a pharmaceutically acceptable coating preventing the release of the active agent in the stomach and allowing the release in the upper part of the intestinal tract.

By particulate form or multiparticles is meant drug particles having an average size of lower than about 3 mm, preferably between about 1 μm to 3 mm.

A preferred group of drug microparticles according to the invention are those having an effective average size of less than about 1000 μm, preferably between about 10 and 800 μM, more preferably between 30 and 200 μm, optionally combined with one or more pharmaceutically acceptable enteric coating ingredients, e.g. as disclosed hereinafter, for example hydroxypropylmethylcellulose phthalate or methacrylic acid copolymers, and a stabilizer, e.g. colloidal silica, to form the microparticles. Such microparticles may be prepared for instance by spray-drying, fluid-bed drying or precipitation techniques, e.g. coacervation techniques, e.g. to separate a liquid phase of a coating material from a polymeric solution and wrapping of that phase as a uniform layer around suspended core particles. The resulting microparticles may be collected by filtration or centrifugation, washed with an appropriate solvent, and subsequently dried by standard techniques such as spray drying or fluidized bed drying. The resulting coated drug microparticles may optionally be combined with a diluent, e.g. as disclosed hereinafter, for example lactose, mannitol or sucrose, a lubricant, e.g. as disclosed hereinafter, for instance magnesium stearate, and dispensed in a capsule or a sachet.

In another embodiment the drug may optionally be combined with a diluent, e.g. as disclosed herein, a binder, e.g. as disclosed hereinafter, e.g. polyvinylpyrrolidone, a hydroxypropyl methylcellulose or sodium carboxymethylcellulose, and formed into granules, e.g. using a technique such as high or low shear granulation, fluid bed granulation or spray drying, to form the granule drug core. The granules obtained may be coated with enteric coating ingredients, e.g. as disclosed hereinafter, and dispensed in a capsule or a sachet. The granule drug core typically has a diameter of from 0.2 to 2 mm, preferably of from 0.5 to 1.4 mm. The amount of drug present in the core may be from 1 to 95% by weight, preferably from 40 to 70% by weight, based on the total weight of the granule drug core, i.e. excluding any coating, if present.

In another embodiment the drug may optionally be combined with one or more pharmaceutically acceptable extrusion aid(s), e.g. microcrystalline cellulose, an ephrit, pregelled starch, etc., binder(s), e.g. as herein disclosed, or diluents, e.g. as herein disclosed, and formed into pellets, e.g. using a technique such as extrusion spheronisation, direct pellitisation/high or low shear granulation, fluid bed granulation or spray drying/melt concealing, to form the pellet drug core. The pellets obtained may be coated with enteric coating ingredients, e.g. as herein disclosed, and dispensed in a capsule or a sachet. The pellet drug core typically has a diameter of from 0.2 to 2 mm, preferably of from 0.5 to 1.4 mm. The amount of drug present in the core may be from 1 to 95% by weight, based on the total weight of the pellet drug core, i.e. excluding any coating, if present.

In another embodiment, the drug optionally in combination with a pharmaceutically acceptable binder, may be layered onto the surface of a pharmaceutically acceptable seed, typically a particle, e.g. a sphere, of sucrose, starch, microcrystalline cellulose or any combination thereof, to form the bead drug core. Such layering may be solution layering or powder layering. Such a pharmaceutically acceptable seed is preferably a non-pareil sugar/starch sphere of 18-20 mesh, 25-30 mesh or 35-40 mesh, most preferably a non-pareil sugar starch sphere of 25-30 mesh. The beads obtained may be coated with enteric coating ingredients, e.g. as herein disclosed and dispensed in a capsule or a sachet or further processed by layering of another drug. The bead drug core typically has a width of diameter of from 0.2 to 2 mm, preferably of from 0.5 to 1.4 mm . The amount of drug present in the core may be from 1 to 95% by weight, based on the total weight of the bead drug core, i.e. excluding any coating, if present.

In a further embodiment, coated microparticles, granules, beads or pellets may optionally be combined with pharmaceutically acceptable ingredients, e.g. a diluent, binder, lubricant, e.g. as herein disclosed, to form tablets and/or minitablets which disintegrate in the mouth, stomach or small intestine, preferably in the stomach, and release, e.g. enteric coated microparticles, pellets or granules. They may also be combined and incorporated in capsules which dissolve in the mouth, stomach or small intestine, preferably in the stomach, and release enteric coated microparticles, pellets or granules.

The term “minitablets” within the scope of this application denotes small tablets with an overall weight of approximately 3 to 10 mg, e.g. approximately 4 to 9 mg, e.g. approximately 7 mg, in their uncoated form. The minitablets may have any shape known to the skilled person for tablets, e.g. round, e.g. with a diameter of about 1.2 to 3 mm, preferably 1.5 to 3 mm; cyclindrical e.g. having a convex upper face and convex lower face, and e.g. with a cylindrical diameter and height independently of each other are from 1 to 3 mm; or biconvex minitablets, e.g. whose height and diameter are approximately equal and are from 1.5 to 3 mm.

Minitablets comprising mycophenolic acid, a salt or a prodrug thereof, e.g. MMF, are preferably of a total weight, i.e. the weight of the tablet core plus the weight of any coating, if present of 3 to 10 mg. The enteric coating, where present, preferably comprises 15 to 50% of the total weight, more preferably 15 to 35%, e.g. 25 to 35% or 15 to 30%.

MPA, a salt thereof, or a prodrug thereof, e.g. MMF, may be granulated prior to the preparation of minitablets. The granulate may be enteric coated prior to the preparation of minitablets, and/or the minitablets may be enteric coated.

In order to provide efficient immunosuppression and to reduce the risk of acute graft rejection to a minimum, it is desirable to combine two or more immunosuppressants having each a different mechanism of action. MPA, their salts and produgs, e.g. MMF, are immunosuppressant drugs known as non-competitive, reversible inhibitors of inosine monophosphate dehydrogenase (IMPDH), therefore inhibiting de novo synthesis of purines and exhibiting a cytostatic effect on lymphocytes. Rapamycin and rapamycin derivatives are immunosuppressant drugs known to inhibit T-cell activation and proliferation. It is therefore desirable to combine these two types of immunosuppressants, e.g. to inhibit graft rejection in transplanted patients, and in particular of maintenance transplanted patients.

Accordingly in order to enhance convenience and compliance of patients, the present invention provides a fixed combination comprising a) mycophenolic acid, a salt thereof, or a prodrug thereof, e.g. MMF and b) rapamycin or a derivative thereof.

Rapamycin is an immunosuppressive lactam macrolide that is produced by Streptomyces hygroscopicus.

A rapamycin derivative is a substituted rapamycin e.g. a 40-O-substituted rapamycin e.g. as described in U.S. Pat. No. 5,258,389, WO 94/09010, WO 92/05179, U.S. Pat. No. 5,118,677, U.S. Pat. No. 5,118,678, U.S. Pat. No. 5,100,883, U.S. Pat. No. 5,151,413, U.S. Pat. No. 5,120,842, WO 93/11130, WO 94/02136, WO 94/02485 and WO 95/14023 all of which are incorporated herein by reference; a 16-O-substituted rapamycin e.g. as disclosed in WO 94/02136, WO 95/16691 and WO 96/41807, the contents of which are incorporated herein by reference; or a 32-hydrogenated rapamycin e.g. as described in WO 96/41807 and U.S. Pat. No. 5,256,790, incorporated herein by reference.

Preferred rapamycin derivatives are compounds of formula I wherein 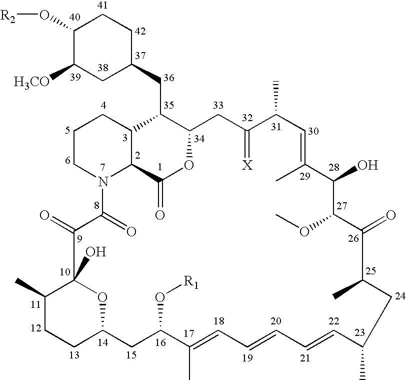
wherein
R₁ is CH₃ or C₃₋₆alkynyl,
R₂ is H or —CH₂—CH₂—OH, 3-hydroxy-2-(hydroxymethyl)-2-methyl-propanoyl or tetrazolyl,
and X is ═O, (H,H) or (H,OH)
provided that R₂ is other than H when X is ═O and R₁ is CH₃,
or a prodrug thereof when R₂ is —CH₂—CH₂—OH, e.g. a physiologically hydrolysable ether thereof.

Particularly preferred rapamycin derivatives of formula I are 40-O-(2-hydroxyethyl)-rapamycin (Compound A hereinafter), 40-[3-hydroxy-2-(hydroxymethyl)-2-methylpropanoate]-rapamycin (also called CCI779), 40-epi-(tetrazolyl)-rapamycin (also called ABT578), 32-deoxorapamycin, or 16-pent-2-ynyloxy-32(S)-dihydro rapamycin. Even more preferred is Compound A.

Rapamycin derivatives also include so-called rapalogs, e.g. as disclosed in WO 98/02441, WO 01/14387 and WO 03/064383, e.g. AP23573, AP23464, AP 23675, AP23841, TAFA-93, biolimus-7 and biolimus-9.

Rapamycin and derivatives thereof have, on the basis of observed activity, e.g. binding to macrophilin-12 (also known as FK-506 binding protein or FKBP-12), e.g. as described in WO 94/09010, WO 95/16691 or WO 96/41807, been found to be useful e.g. as immuno-suppressant, e.g. in the treatment of acute allograft rejection.

Preferably, Compound A, CC1779, ABT578, AP23573, 32-deoxorapamycin, or 16-pent-2-ynyloxy-32(S)-dihydro rapamycin, even more preferably Compound A, and/or a MPA salt or MMF, ever more preferably MPA sodium salt, is used as active ingredient in the fixed combination of the invention.

The term “fixed combination” within the scope of this application denotes combinations wherein MPA, a salt thereof, e.g. sodium mycophenolate salt, or a prodrug thereof, e.g. MMF, and rapamycin or a derivative thereof are formulated in a single administration unit as well as combinations wherein the two active substances are formulated separately in subunits and then combined in a single administration unit.

Accordingly, in another embodiment, the present invention provides a fixed combination wherein two active substances are formulated in subunits separately formulated in each case in the same administration unit.

In yet another embodiment, the present invention provides a fixed combination wherein the two active substances are formulated in a common administration unit without impairing the stability or the release profiles of the two active substances and/or without reducing their bioavailability.

In a further aspect, the present invention provides a fixed combination comprising a) mycophenolic acid, a salt thereof, e.g. sodium mycophenolate salt, or a prodrug thereof, e.g. MMF and b) rapamycin or a derivative thereof, wherein the combination may be formulated so that release of MPA, a salt thereof, e.g. sodium mycophenolate salt, or a prodrug thereof, e.g. MMF, in the stomach is prevented or substantially prevented and MPA, a salt thereof, e.g. sodium mycophenolate salt, or a prodrug thereof, e.g. MMF, is released in the upper part of the intestinal tract. In case of separately formulated subunits, both subunits may be coated, e.g. enteric coated, preferably at least the subunit containing MPA, a salt thereof, e.g. sodium mycophenolate salt, or a prodrug thereof, e.g. MMF, as the active substance is enteric coated.

For the preparation of compositions of the invention comprising rapamycin or a derivative thereof, preferably, rapamycin or a derivative thereof in form of a stabilized powder and/or in form of a solid dispersion is used.

Rapamycin or a derivative thereof may be stabilized, e.g. by mixing with an antioxidant in an amount of up to 1%, more preferably from 0.01 to 0.5% (based on the weight of the rapamycin or rapamycin derivative). Preferred antioxidants are e.g. 2,6-di-tert.-butyl4-methylphenol (hereinafter BHT), vitamin E or C, BHT being particularly preferred. Particularly preferred is a mixture of rapamycin or a derivative thereof and 0.2% (based on the weight of the rapamycin or a derivative thereof) of antioxidant, preferably BHT.

A solid dispersion of rapamycin or a derivative thereof may comprise rapamycin or one of its derivatives and a carrier medium e.g. as disclosed in EP 839028, or e.g. as described below.

Preferably, subunits comprising rapamycin or a derivative thereof, preferably compound A, are coated with a coating material that protects the core against humidity uptake. Suitable coating materials that may protect against humidity uptake of the coated core include e.g. those as known under the tradename Opadry® II (HP) and available from Colorcon, consisting of partially hydrolysed polyvinylalcohol, polyethylene glycol (PEG) 3350, and talc. Preferably, subunits comprising rapamycin or a derivative thereof, e.g. compound A, are coated with a 15% aqueous coating solution to result in 10% dried film by weight of core weight or weight of enteric coated unit.

Fixed combinations according to the present invention may be in the form of e.g. tablets, bi- or tri-layer tablets, dry coated tablets, bull eye tablets, pellets, granules, beads or minitablets.

To achieve the desired overall dose of the active substances per day, a plurality of pellets, granules, beads or minitablets may be required that may e.g. be filled into a suitable container, e.g. capsules, e.g. hard gelatine capsules or e.g. gelatine-free capsules such as hydroxypropyl methylcellulose (HPMC) capsules, or sachets.

In case subunits comprising rapamycin or a derivative thereof, e.g. compound A, are not coated with a coating that protects the core against humidity uptake, the pellets, granules, beads or minitablets are preferably filled into a container wherein they are exposed to an atmosphere that is substantially free of water, e.g. an atmosphere that contains less than about 6% of water. In case said container is a capsule, the capsule shell preferably has a water content of less than about 10%, preferably less than about 6% of water.

Suitable capsules with a capsule shell having a water content of less than about 10%, preferably less than about 4-6% of water, include hydroxypropyl methylcellulose (HPMC) capsules, e.g. such as known under the tradename Quali-V® and available from Shionogi Qualicaps, Inc.

For the fixed combination, an administration unit may comprise e.g. i) a plurality of pellets, granules, beads and/or minitablets containing both active substances, ii) a plurality of pellets, granules, beads and/or minitablets containing one active substance and a plurality of pellets, granules, beads and/or minitablets containing the other active substance, iii) a plurality of pellets, granules, beads and/or minitablets containing one of the two active substances and a tablet containing the other active substance, iv) a tablet containing one active substance and a tablet containing the other active substance, or v) a tablet containing both active substances.

In one embodiment, the present invention provides a composition comprising a) enteric coated or non-enteric coated administration subunits of MPA, a salt thereof, e.g. sodium mycophenolate salt, or a prodrug thereof, e.g. MMF, and b) administration subunits of rapamycin or a derivative thereof, and, wherein the subunits are in the form of tablets, pellets, granules, beads or minitablets. The form of the subunits of a) MPA, a salt thereof, e.g. sodium mycophenolate salt, or a prodrug thereof, e.g. MMF, and b) rapamycin or a derivative thereof, may be different, e.g. the first may be in the form of pellets, granules, beads or minitablets whereas the second may be in the form of tablets or vice versa. Preferably, the subunits of a) MPA, a salt thereof, e.g. sodium mycophenolate salt, or a prodrug thereof, e.g. MMF, are in the form of minitablets and the subunits of b) rapamycin or a derivative thereof, are in the form of a tablet or minitablets.

In another embodiment, the present invention provides a composition comprising a) MPA, a salt thereof, e.g. sodium mycophenolate salt, or a prodrug thereof, e.g. MMF, preferably in form of enteric coated granules, and b) rapamycin or a derivative thereof in a common administration unit in the form of tablets, pellets, granules, beads or minitablets. For example, enteric coated or non-enteric coated granules or pellets of MPA, a salt thereof, e.g. sodium mycophenolate salt, or a prodrug thereof, e.g. MMF, may be mixed with rapamycin or a derivative thereof, or a stabilized powder comprising rapamycin or a derivative thereof, or a solid dispersion of rapamycin or a derivative thereof. Optionally, said mixture may be compressed, e.g. into tablets.

In another embodiment, the present invention provides a composition comprising a core comprising a) MPA, a salt thereof, e.g. sodium mycophenolate salt, or a prodrug thereof, e.g. MMF, and b) rapamycin or a derivative thereof in a common administration unit in the form of tablets, pellets, granules, beads or minitablets wherein the core is optionally enteric coated. Preferably, the core is enteric coated.

In another embodiment, the present invention provides a composition comprising a core comprising MPA, a salt thereof, e.g. sodium mycophenolate salt, or a prodrug thereof, e.g. MMF, an enteric coating, and an additional coating comprising rapamycin or a derivative thereof, e.g. as an overcoat. For example, a suitable coating comprising rapamycin or a rapamycin derivative is in form of a solid dispersion, e.g. in form of a solid dispersion as disclosed in EP 839028, the contents thereof being incorporated herein by reference. The coating comprising rapamycin or a rapamycin derivative is free or substantially free of MPA, a salt thereof, e.g. sodium mycophenolate salt, or a prodrug thereof, e.g. MMF. Optionally, there is yet another coating between the enteric coating and the overcoating comprising rapamycin or a rapamycin derivative. Optionally, there is yet another overcoating upon the overcoating comprising rapamycin or a rapamycin derivative.

In the fixed combination of the invention, MPA, a salt thereof, e.g. sodium mycophenolate salt, or a prodrug thereof, e.g. MMF, preferably is in form of minitablets, pellets, beads or granules.

Preferably, in the fixed combination of the invention the rapamycin or rapamycin derivative is formulated into a tablet or into minitablets and/or the MPA, a salt thereof, e.g. sodium mycophenolate salt, or a prodrug thereof, e.g. MMF, is in form of minitablets. Even more preferably, the fixed combination of the invention is in form of minitablets containing both active substances in each minitablet.

The compositions of the invention, i.e. the compositions comprising MPA, a salt thereof, or a prodrug thereof as the only active ingredient, or rapamycin or rapamycin derivative as the only active ingredient, or a combination of MPA, a salt thereof, or a prodrug thereof and rapamycin or rapamycin derivative, may contain natural and/or artificial auxiliary substances and additives which are commonly used to prepare pharmaceutical compositions. Examples include carriers, fillers, binders, film-building agents, disintegrants, lubricants, diluents, anti caking agents, vitamins, amino acids, fibers, solubilizers, emulsifiers, flavorants, sweeteners, enzymes, buffers, stabilizers, colorants, dyes, antioxidants, anti-adherents, preservatives, glidants and lubricants. Such auxiliary substances and additives are known to those skilled in the art, and thus, only a limited number will be specifically referenced. It will be appreciated that although the excipients are described herein by reference to a particular function, any particular excipient may have alternative or multiple functions, e.g. starches may act as e.g. carrier and/or disintegrant.

A suitable carrier medium for a solid dispersion comprising rapamycin or a rapamycin derivative may comprise a water-soluble polymer, preferably a cellulose derivative such as hydroxypropylmethylcellulose (HPMC), e.g. HMPC with a low apparent viscosity, e.g. below 100 cps as measured at 20° C. for a 2% by weight aqueous solution, e.g. below 50 cps, preferably below 20 cps, for example HPMC 3 cps, hydroxypropylmethylcellulose phthalate, or polyvinylpyrrrolidone (PVP), e.g. a PVP having an average molecular weight between about 8,000 and about 50,000 Daltons; hydroxypropylcellulose (HPC) or a derivative thereof, e.g. HPC having a low dynamic viscosity in aqueous media, e.g. water, e.g. below about 400 cps, e.g. below 150 cps as measured in a 2% aqueous solution at 25° C.; a polyethylene glycol (PEG), e.g. a PEG having an average molecular weight between 1000 and 9000 Daltons, e.g. between about 1800 and 7000 Daltons, for example PEG 2000, PEG 4000 or PEG 6000; a saturated polyglycolised glyceride, e.g. a Gelucir®; or a cyclodextrin, e.g. a β-cyclodextrin or an α-cyclodextrin. The water-soluble polymer, polyethylene glycol, saturated polyglycolised glyceride, or cyclodextrin is present in an amount of up to 99.99% by weight, for example 10 to 95 wt-%, based on the total weight of the solid dispersion.

A carrier medium for a solid dispersion comprising rapamycin or a rapamycin derivative may further comprise a water-soluble or water-insoluble saccharose and/or other acceptable carrier or filler such as lactose, or microcrystalline cellulose.

A carrier medium for a solid dispersion comprising rapamycin or a rapamycin derivative may further comprise one or more surfactants, for example a non-ionic, ionic, anionic or amphoteric surfactant. Examples of suitable surfactants include polyoxyethylene-polyoxypropylene co-polymers and block co-polymers known, for example, under the trade names Pluronic or Poloxamer, e.g. Poloxamer 188; ethoxylated cholesterins, e.g. a Solulan®, e.g. Solulan C24; vitamin derivatives, e.g. vitamin E derivatives such as tocopherol polyethylene glycol succinate (TPGS); sodium dodecylsulfate or sodium laurylsulfate; a bile acid or salt thereof, e.g. cholic acid, glycolic acid or a salt, e.g. sodium cholate; or lecithin.

Preferably, the solid dispersion comprising rapamycin or a rapamycin derivative does not comprise a surfactant. Furthermore, a carrier medium for a solid dispersion comprising rapamycin or a rapamycin derivative may further comprise one or more disintegrants. Examples of disintegrants include Polyplasdone™; sodium starch glycolate; and croscarmelose.

The carrier medium for solid dispersion may further comprise one or more antioxidants, such as ascorbyl palmitate, butyl hydroxyl anisole (BHA), butyl hydroxy toluene (BHT) and tocopherols, may be present in an amount of about 0.05 to about 1% by weight, preferably 0.2 to 0.4% by weight, of the total weight of the solid dispersion and in an amount of about 0.003 to about 0.05% by weight of the total weight of an uncoated composition of the invention.

Accordingly in one embodiment, the present invention provides a composition wherein the rapamycin or a rapamycin derivative is in form of a solid dispersion and wherein the carrier medium for the solid dispersion comprises rapamycin or a rapamycin derivative and one or more excipients selected from
a) a water-soluble polymer, e.g. a HPMC and/or a polyvinylpyrrolidone, or a cyclodextrin,
b) saccharose, a microcrystalline cellulose or lactose,
c) a surfactant, e.g. a polyoxyethylene-polyoxypropylene co-polymer or block co-polymer,
d) a disintegrant, and
e) an antioxidants, e.g. BHT.

Suitable fillers for compositions of the present invention containing a) MPA, a salt thereof, e.g. sodium mycophenolate salt, or a prodrug thereof, e.g. MMF, including also in particulate form as indicated above, and/or b) rapamycin or a rapamycin derivative, e.g. a stabilized rapamycin or rapamycin derivative, or e.g. rapamycin or a rapamycin derivative in form of a solid dispersion comprise e.g. a water-soluble or water-insoluble saccharide such as lactose or mannitol; glucose anhydrate; microcrystalline cellulose, e.g. as known and commercially available under the trade name Avicel® from FMC Corporation; colloidal silicon dioxide, e.g. as known and commercially available under the trade name Aerosil®.

Suitable binders for a composition of the present are polyvinylpyrrolidone (PVP), e.g. PVP K30 or PVP K12, as known and commercially available under the trade name Povidone® from the BASF company; or hydroxypropylmethylcellulose (HPMC), e.g. HMPC with a low apparent viscosity, e.g. below 100 cps as measured at 20° C. for a 2% by weight aqueous solution, e.g. below 50 cps, preferably below 20 cps, for example HPMC 3 cps, as known and commercially available under the name Pharmacoat® 603 from the Shin-Etsu company; or sodium carboxymethylcellulose.

A mixture of excipients may be present. Any excipient, if present, is generally present in an amount of up to about 85%, e.g. about 0.05 to about 85% by weight based on the total weight of the uncoated composition.

MPA, a salt thereof, e.g. sodium mycophenolate salt, or a prodrug thereof, e.g. MMF, may be granulated in the presence of a suitable filler and binder, e.g. as mentioned in the preceding paragraph, prior to the preparation of the combinations of the invention.

Suitable disintegrants for compositions of the present invention containing a) MPA, a salt thereof, e.g. sodium mycophenolate salt, or a prodrug thereof, e.g. MMF, including also in particulate form, and/or b) rapamycin or a rapamycin derivative, e.g. a stabilized rapamycin or rapamycin derivative, or e.g. rapamycin or a rapamycin derivative in form of a solid dispersion include excipients which facilitate the disintegration of a solid dosage form, e.g. a tablet or minitablet, when placed in an aqueous environment. Examples of suitable disintegrants include natural starches, such as i) maize starch, potato starch, and the like, ii) directly compressible starches, e.g. Sta-rx® 1500, modified starches, starch derivatives such as e.g. carboxymethyl starches and sodium starch glycolate, available as Primojel®, Explotab®, Explosol®, and iii) ephrit; crosslinked polyvinylpyrrolidones, e.g. crospovidones, e.g. Polyplasdone® XL and Kollidon® CL; alginic acid or sodium alginate; methacrylic acid-divinylbenzene copolymer salts, e.g. Amberlite® IRP-88; and cross-linked sodium carboxymethylcellulose, available as e.g. Ac-di-sol®, Primellose®, Pharmacel® XL, Explocel®, and Nymcel® ZSX, or a mixture thereof. The disintegrant or disintegrants may be present in an amount of 1 to 20%, e.g. 5 to 15% by weight of the total weight of an uncoated composition of the invention.

Suitable lubricants, e.g. magnesium stearate, talc, hydrogenated castor oil, glycerine monostearate or sodium fumarylstearate may be present in an amount of about 0.1% to about 3% by weight of the total weight of an uncoated composition of the invention.

The preferred enteric coating for compositions comprising MPA, a salt or a prodrug of MPA comprises a film-forming agent selected from e.g. cellulose acetate phthalate; cellulose acetate trimelitate; methacrylic acid copolymers, e.g. copolymers derived from methylacrylic acid and esters thereof, containing at least 40% methylacrylic acid; hydroxypropyl methylcellulose phthalate; and hydroxypropylmethylcellulose acetate succinate.

Typical cellulose acetate phthalates have an acetyl content of 17-26% and a phthalate content of from 30-40% with a viscosity of ca. 45-90 cP. An example of an appropriate cellulose acetate phthalate is the marketed product CAP (Eastman Kodak, Rochester N.Y., USA).

Typical cellulose acetate trimellitates have an acetyl content of 17-26%, a trimellityl content from 25-35% with a viscosity of ca. 15-20 cS. An example of an appropriate cellulose acetate trimellitate is the marketed product CAT (Eastman Kodak Company, USA).

Methacryclic acid copolymers include preferably copolymers derived from methylacrylic acid and esters thereof, containing at least 40% methylacrylic acid, more preferably those of molecular weight above 100,000 Daltons based on, e.g. methylacrylate and methyl or ethyl methylacrylate in a ratio of about 1:1. Typical products include Eudragit L, e.g. L 100-55, marketed by Rohm GmbH, Darmstadt, Germany.

Hydroxypropyl methylcellulose phthalates, typically have a molecular weight of from 20,000 to 100,000 Daltons e.g. 80,000 to 130,000 Daltons, e.g. a hydroxypropyl content of from 5 to 10%, a methoxy content of from 18 to 24% and a phthalyl content from 21 to 35%. Examples of suitable hydroxypropyl methylcellulose phthalates are the marketed products having a hydroxypropyl content of from 6-10%, a methoxy content of from 20-24%, a phthalyl content of from 21-27%, a molecular weight of about 84,000 Daltons known under the trade mark HP50 and available from Shin-Etsu Chemical Co. Ltd., Tokyo, Japan, and having a hydroxypropyl content, a methoxy content, and a phthalyl content of 5-9%, 18-22% and 27-35%, respectively, and a molecular weight of 78,000 Daltons, known under the trademark HP55 and available from the same supplier.

Examples of suitable hydroxypropylmethylcellulose acetate succinate may be used as known under the trademark Aqoat LF or Aqoat MF and commercially available, e.g. from Shin-Etsu Chemical Co. Ltd., Tokyo, Japan.

The enteric coating may further comprise further components such as plasticizers, e.g. triacetine, triethyl citrate, diethyl sebacate, dibutyl sebacate, polyethyleneglycol 3000, 4000 or 6000, acetyltriethylcitrate, acetyltributylcitrate, or diethylphthalate, and/or antisticking agents, e.g. colloidal silicon dioxide, an synthetic amorphous silicic acid such as Syloid 244 FP, talc, glycerine monostearate, or a sebacic acid diester, e.g. sebacic dibutyl ester. The coating may further comprise, especially in aqueous dispersions, one or more thickening agents to avoid sedimentation of suspended excipients, e.g. HPMC 3 cps or HPMC 6 cps.

Excipients and coatings as described herein for compositions comprising MPA, a salt or a prodrug thereof are suitable for compositions comprising MPA, a salt or a prodrug thereof as the only active ingredient, or also in the fixed combinations.

Reference is made to the extensive literature on the subject for these and other excipients and procedures mentioned herein, see in particular “Handbook of Pharmaceutical Excipients”, Second Edition, edited by Ainley Wade and Paul J. Weller, American Pharmaceutical Association, Washington, USA and Pharmaceutical Press, London; and “Lexikon der Hilfsstoffe für Pharmazie, Kosmetik and angrenzende Gebiete” edited by H. P. Fiedler, 4^{th} Edition, Editio Cantor, Aulendorf and earlier editions which are incorporated herein by reference.

Preferably the enteric coated minitablets, pellets, beads or granules of the invention may further comprise a subcoating. The subcoating is a layer located between the enteric coating and the core, which may act to improve gastric resistance (e.g. reduce acid uptake in the stomach), and/or improve the chemical stability of the core, isolating the core from the enteric coating and/or by reducing the water/solvent uptake during coating.

Suitable materials for said subcoating include hydroxypropyl methyl cellulose (HPMC), e.g. HPMC 3 cps, ethylcellulose, e.g. as 30% aqueous dispersion, e.g. Aquacoat® ECD, and/or mixtures thereof e.g. a mixture of wherein the ratio of HPMC 3cps:ethylcellulose is from 1:1 to 3:1, partially hydrolysed polyvinylalcohol. The subcoating may further comprise one or more further components as described for the enteric coating above, e.g. plasticizers or antisticking agents. In one embodiment the subcoating comprises partially hydrolysed polyvinylalcohol, PEG3350 (as plasticizer) and talc (as antisticking agent), e.g. as commercially available under the tradename Opadry II HP® from Colorcon.

In one embodiment, the present invention provides a composition comprising MMF, MPA or sodium mycophenolate salt in the form of minitablets, pellets, beads or granules. The MMF-, MPA- or sodium mycophenolate-containing minitablets, pellets, beads or granules are preferably enteric coated. Preferred minitablets, pellets, beads or granules comprise:
a) MMF, MPA or sodium mycophenolate; and
b) one or more excipients selected from:
   (i) a binder;
   (ii) a filler;
   (iii) a disintegrant; and
   (iv) a lubricant.

More preferably the MMF- or sodium mycophenolate-containing minitablets, pellets, beads or granules comprise in addition to the drug substance, a binder, a filler, a disintegrant and a lubricant.

MMF, MPA or sodium mycophenolate is preferably present in the minitablets, pellets, beads, microparticles or granules in an amount of 1 to 95% by weight, based on the total weight of the tablet core, i.e. excluding any coating if present, more preferably 20 to 80%, most preferably 40 to 70%.

The MMF-, MPA- or sodium mycophenolate-containing minitablets are preferably of a total weight (i.e. the weight of the tablet core plus the weight of any coating, if present) of 3 to 14 mg. The enteric coating, where present, preferably comprises 15 to 50% of the total weight, more preferably 15 to 35%, e.g. 25 to 35% or 15 to 30%.

The MMF-, MPA or sodium mycophenolate-containing minitablets, pellets, beads or granules may contain one or more binders, e.g. as defined above. In preferred embodiments the binder comprises (i) polyvinylpyrrolidone, more preferably PVP K30, and/or (ii) HPMC, more preferably HPMC with a low apparent viscosity, e.g. below 100 cps as measured at 20° C. for a 2% by weight aqueous solution, e.g. below 50 cps, preferably below 20 cps, most preferably HPMC 3 cps. Preferably the MMF-containing minitablets, pellets, beads or granules comprise the binder in an amount of 1 to 30% by weight, based on the total weight of the drug core, i.e. excluding any coating, if present, more preferably 1 to 20% by weight, most preferably 5 to 15% by weight.

The MMF-, MPA- or sodium mycophenolate-containing minitablets, pellets, beads or granules may contain one or more fillers, e.g. as defined above. Preferably the filler comprises cellulose, more preferably microcrystalline cellulose. The MMF- or sodium mycophenolate-containing minitablets, pellets or granules preferably comprise the filler in an amount of 10 to 90% by weight, based on the total weight of the drug core, more preferably 10 to 50% by weight, most preferably 15 to 35% by weight.

The MMF-, MPA- or sodium mycophenolate-containing minitablets, pellets, beads or granules may contain one or more disintegrants, e.g. as defined above. In preferred embodiments the disintegrant comprises a modified starch or modified cellulose polymer.

Croscarmellose sodium is preferred as a disintegrant. The MMF-, MPA or sodium mycophenolate salt-containing minitablets, pellets, beads or granules preferably comprise the disintegrant in an amount of 1 to 20% by weight, based on the total weight of the drug core, more preferably 5 to 15%.

The MMF-, MPA or sodium mycophenolate-containing minitablets, pellets, beads or granules may contain one or more lubricants, e.g. as defined above, more preferably magnesium stearate, e.g. in an amount of 0.1 to 3% by weight, based on the total weight of the uncoated composition.

Preferably the minitablets, pellets, beads or granules comprising MMF, MPA or sodium mycophenolate are enteric coated, e.g. using one of the enteric coatings described above. More preferred enteric coatings for minitablets, pellets, beads or granules comprising MMF, MPA or sodium mycophenolate comprise:
a) one or more film-forming agents; e.g. added by layers or as a mixture;
and optionally b) a plasticizer;
and optionally c) an anti-sticking agent.

Most preferably the enteric coating for a MMF-, MPA- or sodium mycophenolate-containing minitablet comprises a film-forming agent, a plasticizer and an anti-sticking agent.

The film-forming agent in the enteric coating of MMF-, MPA- or sodium mycophenolate-containing minitablets, pellets, beads or granules may comprise any of those described above, e.g. cellulose acetate phthalate, cellulose acetate trimellitate, methacrylic acid copolymer, hydroxypropyl methylcellulose phthalate or hydroxypropylmethylcellulose acetate succinate. Preferred film-forming agents for MMF-, MPA or sodium mycophenolate-containing minitablets, pellets, beads or granules include methacryclic acid copolymers, hydroxypropyl methylcellulose phthalate and hydroxypropylmethylcellulose acetate succinate.

The MMF-, MPA- or sodium mycophenolate-containing minitablets, pellets, beads or granules preferably comprise an enteric coating comprising the film-forming agent in an amount of 50 to 95% by weight, based on the total weight of the enteric coating, more preferably 60 to 80% by weight.

The plasticizer in the enteric coating of MMF-, MPA- or sodium mycophenolate-containing minitablets, pellets, beads or granules may comprise any of those described above, more preferably triacetine, triethylcitrate or a sebacic acid diester, e.g. diethyl sebacate or dibutyl sebacate. Preferably the plasticizer is present in an amount of 1 to 50% by weight, more preferably 5 to 25%, based on the total weight of the enteric coating.

The anti-sticking agent in the enteric coating of MMF-, MPA- or sodium mycophenolate-containing, pellets, beads or granules may comprise any of those described above, e.g. colloidal silicon dioxide, a synthetic amorphous silicic acid such as Syloid 244 FP, talc, or glycerine monostearate. Preferably the anti-sticking agent is present in an amount of 1 to 50% by weight, more preferably 5 to 25%, based on the total weight of the enteric coating.

Procedures which may be used to prepare and/or to coat the compositions of the invention may be conventional or known in the art or based on such procedures e.g. those described in L. Lachman et al. The Theory and Practice of Industrial Pharmacy, 3^{rd} Ed, 1986, H. Sucker et al, Pharmazeutische Technologie, Thieme, 1991, Hager's Handbuch der pharmazeutischen Praxis, 4^{th} Ed. (Springer Verlag, 1971) and Remington's Pharmaceutical Sciences, 13^{th} Ed., (Mack Publ., Co., 1970) or later editions. Minitablets may e.g. be manufactured on a standard rotary tabletting machine.

The compressibility of a composition comprising a) rapamycin or a rapamycin derivative and b) MPA, MPA salt, e.g. sodium mycophenolate salt, or MPA prodrug, e.g. MMF, formulated in a common administration unit may be enhanced in comparison to the compressibility of either drug alone.

Preferably, the compositions of the invention are protected against light, humidity and oxygen, e.g. by packaging into aluminum foilbags or aluminum blisters.

The compositions of the invention are stable upon storage of the compositions e.g. for 4 weeks at −20° or 50° C. and for 6 and 12 months at 25° C.

The compositions of the invention are useful as immunosuppressants as indicated by standard tests.

The activity and characteristics of the compositions of the invention may be indicated in standard clinical trials.

The compositions of the invention lead to a inter- and intra-patient reduced variability of MPA, MPA salt, for example sodium mycophenolate, or MPA prodrug, for example MMF, e.g. the food effect is reduced. The compositions of the invention may have a beneficial effect as regards the GI side-effects of MPA.

The compositions and combinations of the invention are particularly useful for the following conditions:
a) Treatment or prevention of native or transgenic organ, tissue or cellular allograft or xenograft transplant rejection, e.g. for the treatment of recipients of e.g. heart, lung, combined heart-lung, liver, kidney, pancreatic, skin, pancreatic islet cell, neural cell or corneal transplant; including treatment and prevention of acute rejection; and treatment and prevention of chronic rejection, e.g. as associated with graft-vessel disease. The compositions of the invention are also indicated for the treatment and prevention of graft-versus-host disease, such as following bone marrow transplantation.
b) Treatment and prevention of autoimmune diseases, e.g. immune-mediated diseases and inflammatory conditions, in particular inflammatory conditions with an etiology including an immunological component such as arthritis (for example rheumatoid arthritis, arthritis chronica progrediente and arthritis deformans) and rheumatic diseases. Specific immune-mediated diseases for which the compositions of the invention may be employed include, autoimmune hematological disorders, including, but not limited to hemolytic anaemia, aplastic anaemia, pure red cell anaemia and idiopathic thrombocytopenia), systemic lupus erythematosus, polychondritis, sclerodoma, Wegener granulosis, dermatomyositis, poly-myositis, chronic active hepatitis, primary bilary cirrhosis, myasthenia gravis, psoriasis, Steven-Johnson syndrome, pemphigus, idiophatic sprue, inflammatory bowel diseases (including e.g. ulcerative colitis and Crohn's disease), endocrine ophthalmophathy, Graves disease, sarcoidosis, multiple sclerosis, juvenile diabetes (diabetes mellitus type I), non-infectious uveitis (anterior and posterior), keratoconjunctvitis sicca and vernal keratoconjunctivitis, interstitial lung fibrosis, psoriatic arthritis, vasculitis, glomerulone-phritides (with and without ephritic syndrome, e.g. including idiophatic ephritic syndrome or minimal change nephropathy) and juvenile dermatomyositis.

In particular, the present combinations of the invention are useful for the treatment and prevention of acute or chronic rejection, preferably in maintenance patients.

The dose of rapamycin or the derivative thereof will, of course, vary depending on a variety of factors, for example the compound chosen, the particular condition to be treated and the desired effect. In general, however, satisfactory results are achieved on administration of rapamycin or a derivative thereof at daily dosage rates of the order of ca. 0.1 to 25 mg rapamycin or rapamycin derivative per day, e.g. about 0.1 to 15 mg, about 0.5 to 3 mg, e.g. 0.75 mg, 1 mg, 1.5 mg, 2 mg, or 3 mg per day, administered as a single dose or in divided doses, preferably about 1 mg, 1.5 mg or 2 mg twice a day.

The dose of the MPA, MPA salt, e.g. sodium mycophenolate salt, or MPA prodrug, e.g. MMF, may vary depending on a variety of factors, for example the compound chosen, the particular condition to be treated and the desired effect. In general satisfactory results are obtained on administration e.g. orally at daily dosages on the order of e.g. from about 50 mg to about 2.5 g MPA per day, e.g. about 250 mg to about 2.2 g MPA, e.g. about 360 mg, about 720 mg, about 740 mg, about 1.1 g, about 1.5 g, about 2.2 g, administered as a single dose or in divided doses, preferably about 360 mg to 720 mg MPA twice a day. Dosages of MPA salt or prodrug are to be calculated to correspond to the above mentioned dosages of MPA.

Accordingly, the present invention provides a fixed combination to be administered twice a day comprising a) rapamycin or a rapamycin derivative in an amount of ca. 0.1 to 25 mg, e.g. about 1 to 3 mg, and b) mycophenolic acid, a salt thereof or a prodrug thereof, e.g. MMF in an amount of corresponding to ca. 50 mg to 2.5 g MPA, e.g. to about 360 mg to 1.5 g MPA, preferably a combination to be administered twice a day comprising a) rapamycin or a rapamycin derivative in an amount of about 1 mg, 1.5 mg or 2 mg, and b) mycophenolic acid, a salt thereof or a prodrug thereof, e.g. MMF in an amount of corresponding to about 360 to 720 mg MPA.

The following examples illustrate various aspects of the invention.

### EXAMPLE 1

### Composition of Enteric Coated Sodium Mycophenolate Minitablets

Minitablets of sodium mycophenolate are prepared by granulation of sodium mycophenolate, Aerosil 200 and Povidone (PVP) K30 with Ethanol 94% for granulation in an amount as indicated in Table 1. After grinding, drying and sieving, the granulate is mixed with the other ingredients as given in Table 1 at dry stage and compressed into minitablets. The resultant minitablets finally are coated with an aqueous dispersion of the coating ingredients (Coating 1) or with an organic solution of the coating ingredients (Coating 2) as given in Table 1.

TABLE 1
Compositions of a minitablet of sodium
mycophenolate (amounts given in mg)

| | Core | | | |
|---|---|---|---|---|
| | A | B | C | D |
| Sodium mycophenolate | 4.810 | 4.748 | 4.810 | 4.748 |
| Povidone K-30 | 0.500 | 0.494 | 0.500 | 0.494 |
| Aerosil 200 | 0.165 | 0.163 | 0.165 | 0.163 |
| Ethanol 94% for granulation | q.s. | q.s. | q.s. | q.s. |
| Hydroxypropyl methyl cellulose | 0.138 | 0.136 | 0.138 | 0.136 |
| Lactose, anhydrous | 1.006 | 0.993 | 1.006 | 0.993 |
| Starch Sta RX | 0.210 | 0.207 | 0.210 | 0.207 |
| Crospovidone | 0.766 | 0.756 | 0.766 | 0.756 |
| Magnesium stearate | 0.155 | 0.153 | 0.155 | 0.153 |
| Total Core | 7.750 | 7.650 | 7.750 | 7.650 |

Coating 1

| | | | | |
|---|---|---|---|---|
| Eudragit L 30 D (dry) | 2.325 | 2.325 | — | — |
| Triacetine | 0.233 | 0.233 | — | — |
| Syloid 244 FP | 0.543 | 0.543 | — | — |
| Water | q.s. | q.s. | — | — |

Coating 2

| | | | | |
|---|---|---|---|---|
| HP 50 (dry) | — | — | 2.325 | 2.325 |
| Triethylcitrate | — | — | 0.233 | 0.233 |
| Colloidal silicon dioxide | — | — | 0.692 | 0.692 |
| Acetone, Ethanol 94% 1:1 | — | — | q.s. | q.s. |
| Total (Core plus Coating) | 10.850 | 10.750 | 11.000 | 10.900 |

Alternatively, minitablets may be coated with an organic solution of Eudragit L100-55 instead of an aqueous dispersion of Eudragit L 30 D.

Hard gelatine capsules of size 0 or HPMC capsules of size 0 are filled with 40 minitablets of sodium mycophenolate of composition A or C in a suitable encapsulating machine.

### EXAMPLE 2

### Fixed Combination of Enteric Coated Sodium Mycophenolate Minitablets and Compound A Minitablets

Minitablets of Compound A or rapamycin are prepared by mixing the solid dispersion of Compound A with the other ingredients as given in Table 3 (composition a or b) at dry stage and compression to minitablets.

The minitablets of Compound A or rapamycin are optionally coated with an aqueous coating under dry coating conditions with a hot and slow pumping rate with the protective coat as given in Table 4. The loss on drying of the coated minitablets is less than 2%.

The 2% solid dispersion is prepared by dissolving Compound A and dispersing the carrier medium as given in Table 2 in an ethanol/acetone mixture. The solvents are then evaporated, and the resulting dry residue is milled.

TABLE 2
Composition of solid dispersions of Compound A (amounts given in %)

| | 9.09% solid | 2% solid |
|---|---|---|
| Composition | dispersion | dispersion |
| Compound A or rapamycin | 9.09 | 2.0 |
| Hydroxypropylmethylcellulose 3 cps | 81.82 | 80.0 |
| Lactose 200 mesh | 8.89 | 17.8 |
| Butylated hydroxy toluene | 0.20 | 0.2 |

TABLE 3
Composition of a minitablet of Compound A (amounts given in mg)

| | a | b |
|---|---|---|
| 2% Solid dispersion of Compound A (Table 2) | 1.280 | 1.280 |
| or rapamycin | | |
| Lactose, anhydrous | 3.808 | 4.416 |
| Crospovidone | 1.280 | 0.640 |
| Magnesium stearate | 0.032 | 0.064 |
| Total | 6.40 | 6.40 |

TABLE 4
Coating (amounts given in mg)

| | |
|---|---|
| Opadry II HP, 85F29116. clear | 0.64 |
| Water | Q s. |
| Kernel compound A/(Table 3) | 6.4 |
| Coated minitablet compound A | 7.04 |

Minitablets of sodium mycophenolate are prepared by granulation of sodium mycophenolate, Aerosil 200 and PVP K30 with Ethanol 94% for granulation as indicated in Table 1 (composition B or D). After grinding, drying and sieving, the granulate is mixed with the other ingredients as given in Table 1 (composition B or D) at dry stage and compressed into minitablets. The resultant minitablets finally are coated with an aqueous dispersion of the coating ingredients (Coating 1) or with an organic solution of the coating ingredients (Coating 2) as given in Table 1.

Subunits may be filled into HPMC capsules or hard gelatine capsules, preferably in HPMC capsules with low water content.

For example, HPMC capsules of size 0 are filled with 27 sodium mycophenolate minitablets and 13 uncoated minitablets of Compound A in a suitable encapsulating machine.

### EXAMPLE 3

### Fixed Combination of Enteric Coated Sodium Mycophenolate Minitablets and a Compound A Tablet

Tablets of Compound A are prepared by mixing the solid dispersion of Compound A with the other ingredients as given in Table 5 at dry stage and compression to tablets.

The 9.09% solid dispersion is prepared by dissolving Compound A and dispersing the carrier medium as given in Table 2 in an ethanol/acetone mixture. The solvents are then evaporated, and the resulting dry residue is milled.

TABLE 5
Composition of a tablet of Compound A (amounts given in mg)

| | a | b | c | d |
|---|---|---|---|---|
| 9.09% Solid dispersion of | 5.5 | 5.5 | 5.5 | 5.5 |
| Compound A (Table 2) | | | | |
| Lactose, anhydrous | 63.6 | 65.7 | 34.25 | 39.0 |
| Crospovidone | 16.0 | 8.0 | 10.0 | 5.0 |
| Magnesium stearate | 0.4 | 0.8 | 0.25 | 0.50 |
| Total | 80.0 | 80.0 | 50.0 | 50.0 |

Minitablets of sodium mycophenolate are prepared by granulation of the sodium mycophenolate, Aerosil 200 and PVP K30 with Ethanol 94% for granulation as indicated for formulations A and C in Table 1. After grinding, drying and sieving, the granulate is mixed with the other ingredients as given in Table 1 (composition A or C) at dry stage and compressed into minitablets. The resultant minitablets finally are coated with an aqueous dispersion of the coating ingredients (Coating 1) or with an organic solution of the coating ingredients (Coating 2) as given in Table 1.

HPMC capsules of size 0 are filled with 40 sodium mycophenolate minitablets and 1 tablet of Compound A in a suitable encapsulating machine.

### EXAMPLE 4

### Fixed Combination of Enteric Coated Sodium Mycophenolate Minitablets and a Coated Compound A Tablet

Tablets of Compound A are prepared as described in Example 3. The tablets of Compound A according to compositions b or d of Table 5 are then coated with 10% (film dry) of kernel weight as given in Table 6. The tablets of Compound A are optionally coated with an aqueous coating under dry coating conditions with a hot and slow pumping rate with the protective coat as given in Table 6. The loss on drying of the coated minitablets is not more than 2%.

TABLE 6
Coating of a tablet of Compound A (amounts given in mg)

| | | |
|---|---|---|
| Compound A Tablet of Example 3 | 80.0 | 50.0 |
| (column b or d) | | |
| Opadry II HP (85F29116. clear) | 8.000 | 5.000 |
| Water | qs | qs |
| Total core + coating | 88.000 | 55.000 |

Minitablets of sodium mycophenolate are prepared and coated as described in Example 3.

Hard gelatine capsules of size 0 elongated or 00 or HPMC capsules of size 0 elongated or 00 are filled with 40 sodium mycophenolate minitablets and 1 tablet of Compound A in a suitable encapsulating machine.

### EXAMPLE 5

### Fixed Combination Comprising Enteric Coated Minitablets Comprising Sodium Mycophenolate and Compound A

Minitablets of sodium mycophenolate and Compound A are prepared by mixing the sodium mycophenolate granulate, the solid dispersion of Compound A and the other ingredients as given in Table 7 at dry stage and compressing into minitablets. The sodium mycophenolate granulate is manufactured by granulating the sodium mycophenolate, Aerosil 200 and PVP K30 with Ethanol 94%, the granules are grinded, dried and sieved before mixing with the other ingredients. Finally, the minitablets are coated with an organic solution of Eudragit L100-55 as given in Table 7, Coating 1, or with an organic solution of the coating ingredients as given in Table 7, Coating 2 or Coating 3.

TABLE 7
Composition of a minitablet comprising sodium mycophenolate and
Compound A (amounts given in mg)
Core

| | |
|---|---|
| Sodium mycophenolate | 4.810 |
| Povidone K-30 | 0.500 |
| Aerosil 200 | 0.165 |
| Ethanol 94% for granulation | q.s. |
| 9.09% Solid dispersion of Compound A | 0.138 |
| Lactose, anhydrous | 1.006 |
| Starch Sta RX | 0.210 |
| Crospovidone | 0.766 |
| Magnesium stearate | 0.155 |
| Total Core | 7.750 |

Coating 1

| | |
|---|---|
| Eudragit L 100-55 (dry) | 2.325 |
| Triacetine | 0.233 |
| Syloid 244 FP | 0.543 |
| Isopropanol, Water 97:3 | q.s. |
| Total (Core plus Coating) | 10.850 |

Coating 2

| | |
|---|---|
| HP 50 (dry) | 2.325 |
| Triethylcitrate | 0.233 |
| Talc | 0.543 |
| Acetone, Ethanol 94% 1:1 | q.s. |
| Total (Core plus Coating) | 10.850 |

Coating 3

| | |
|---|---|
| HP 50 (dry) | 2.325 |
| Dibutyl sebacate | 0.233 |
| Colloidal silicon dioxide | 0.692 |
| Acetone, Ethanol 94% 1:1 | q.s. |
| Total (Core plus Coating) | 11.000 |

HPMC capsules of size 0 are then filled with 40 minitablets in a suitable encapsulating machine.

### EXAMPLE 6

### Fixed Combination Comprising Enteric Coated Minitablets Comprising Sodium Mycophenolate and Compound A with Subsequent Additional Overcoating

Minitablets of sodium mycophenolate and Compound A are prepared as described in Example 5. Finally an overcoat consisting of 10% (kernel weight) Opadry II is added (see Table 8). The minitablets of sodium mycophenolate and Compound A are optionally coated with an aqueous coating under dry coating conditions with a hot and slow pumping rate with the protective coat as given in Table 6. The loss on drying of the coated minitablets is not more than 2%.

TABLE 8
Overcoat of coated minitablets comprising sodium mycophenolate and
Compound A (amounts given in mg)

| | |
|---|---|
| Enteric coated core of Example 5 coated with | 11.000 |
| coating 3 of example 5 | |
| Opadry II HP (85F29116. clear) | 1.100 |
| Water | q.s. |
| Total (Core plus Coatings) | 12.100 |

Hard gelatine capsules of size 00 or HPMC capsules Sie 00 are then filled with 40 minitablets in a suitable encapsulating machine.

### EXAMPLE 7

### Fixed Combination Comprising Enteric Coated Minitablets Comprising Sodium Mycophenolate Coated with Compound A

Minitablets of sodium mycophenolate are prepared by granulation of sodium mycophenolate, Aerosil 200 and PVP K30 with Ethanol 94% for granulation. After grinding, drying and sieving, the granulate is mixed with the other ingredients as given in Table 1 (composition A or C) at dry stage and compressed into minitablets. The resultant minitablets are coated with an aqueous dispersion of the coating ingredients (Coating 1) or with an organic solution of the coating ingredients (Coating 2) as given in Table 1.

The resultant coated minitablets are overcoated with a swollen dispersion of the coating ingredients as given in Table 9.

TABLE 9
Coating Composition comprising Compound A (amounts given in mg)

| | | |
|---|---|---|
| Hydroxypropylmethylcellulose | 0.125 | 0.585 |
| Compound A | 0.013 | 0.013 |
| Butylated hydroxy toluene | 0.00025 | 0.0025 |
| Triethylcitrate | | 0.025 |
| Ethanol/Acetone 1:1 | q.s. | q.s. |

The coated minitablets have a total weight of 10.987 mg or 11.475 mg.

HPMC capsules of size 0 are then filled with 40 minitablets in a suitable encapsulating machine.

### EXAMPLE 8

### Fixed Combination Comprising Enteric Coated Minitablets Comprising Sodium Mycophenolate Coated with Compound A

Minitablets of sodium mycophenolate are prepared and coated as described in Example 7. The coated minitablets have a total weight of 11.476 mg or 11.623 mg.

40 layered minitablets are filled into HPMC capsules (size 00) or additionally coated with Opadry II (as given in Table 10) before filling into hard gelatine capsules (size 00) in a suitable encapsulating machine.

TABLE 10
Overcoat (amounts given in mg)

| | | |
|---|---|---|
| enteric coated minitablets comprising sodium | 11.623 | 11.476 |
| mycophenolate coated with Compound A | | |
| Opadry II HP (85F29116. clear) | 1.160 | 1.150 |
| Water | Q.s. | q.s. |
| Total (Core plus Coatings) | 12.783 | 12.626 |

### EXAMPLE 9

In further examples, minitablets comprising sodium mycophenolate or mycophenolate mofetil are prepared as described in Example 1, wherein the core consists of the following components:

TABLE 11
Compositions of a minitablet of sodium mycophenolate
(amounts given in mg)

Core

| | A | B | C | D |
|---|---|---|---|---|
| Sodium mycophenolate | 3.103 | 3.103 | 3.103 | 3.103 |
| Povidone (K-30) | 0.323 | 0.323 | 0.323 | 0.323 |
| Silica colloidal anhydrous | 0.106 | 0.106 | 0.106 | 0.106 |
| Lactose anhydrous | 0.726 | 0.892 | 0.750 | — |
| microcrystalline cellulose | — | — | — | 0.750 |
| Maize starch | 0.166 | — | — | — |
| Crospovidone | 0.524 | 0.501 | — | — |
| Croscarmellose sodium | — | — | 0.643 | 0.643 |
| Magnesium stearate | 0.053 | 0.075 | 0.075 | 0.075 |
| Total core | 5.000 | 5.000 | 5.000 | 5.000 |

TABLE 12
Compositions of a minitablet of sodium
mycophenolate (amounts given in mg)

Core

| | E | F | G | H |
|---|---|---|---|---|
| Sodium mycophenolate | 4.810 | 4.810 | 4.810 | 4.810 |
| Povidone (K-30) | 0.375 | 0.375 | 0.563 | 0.563 |
| Silica colloidal anhydrous | 0.075 | — | 0.075 | — |
| microcrystalline cellulose | 1.377 | 1.452 | 0.940 | 1.015 |
| Crospovidone | — | 0.750 | — | 1.000 |
| Croscarmellose sodium | 0.750 | — | 1.000 | — |
| Magnesium stearate | 0.113 | 0.113 | 0.113 | 0.113 |
| Total core | 7.500 | 7.500 | 7.500 | 7.500 |

TABLE 13
Compositions of a minitablet of mycophenolate
mofetil (amounts given in mg)

| | Core | | | |
|---|---|---|---|---|
| | I | J | K | L |
| Mycophenolate mofetil | 4.060 | 4.060 | 4.060 | 4.060 |
| Povidone (K-30) | 0.375 | 0.375 | 0.563 | 0.563 |
| microcrystalline cellulose | 2.202 | 1.607 | 1.607 | 1.764 |
| Hydroxypropylmethylcellulose 3 cps | — | 0.345 | 0.407 | — |
| Croscarmellose sodium | 0.750 | 1.000 | 0.750 | 1.000 |
| Magnesium stearate | 0.113 | 0.113 | 0.113 | 0.113 |
| Total core weight | 7.500 | 7.500 | 7.500 | 7.500 |

The minitablets containing a core A-L as defined in Table 11, 12 or 13 are coated using one of the following coatings (amounts given in mg):

TABLE 14
Coatings (amounts given in mg)

| Coating | a | b |
|---|---|---|
| Hydroxypropylmethylcellulose phthalate | 1.500 | 2.250 |
| Triethylcitrate | 0.150 | 0.225 |
| Colloidal silicon dioxide | 0.450 | 0.675 |
| Ethanol/acetone 1:1 | q.s. | q.s. |
| Total (coating) | 2.100 | 3.150 |
| Core | 5.000 | 7.500 |
| Total (Core plus Coating) | 7.100 | 10.650 |

TABLE 15
Coatings (amounts given in mg)

| Coating | c | d |
|---|---|---|
| Hydroxypropylmethylcellulose phthalate | 1.500 | 2.250 |
| Diethylsebacate | 0.150 | 0.225 |
| Talc | 0.450 | 0.675 |
| Ethanol/acetone 1:1 | q.s. | q.s. |
| Total (coating) | 2.100 | 3.150 |
| Core | 5.000 | 7.500 |
| Total (Core plus Coating) | 7.100 | 10.650 |

TABLE 16
Coatings (amounts given in mg)

| Coating | e | f |
|---|---|---|
| Eudragit L 30 D aqueous dispersion | 1.500 | 2.250 |
| (30%) - (dry) | | |
| Triethylcitrate | 0.300 | 0.450 |
| Talc | 0.200 | 0.300 |
| Water | q.s. | q.s. |
| Subcoating | | |
| Hydroxypropylmethylcellulose 3 cps | 0.250 | 0.375 |
| Triethylcitrate | 0.025 | 0.038 |
| Talc | 0.035 | 0.052 |
| Water | q.s. | q.s. |
| Total (coating plus subcoating) | 2.310 | 3.465 |
| Core | 5.000 | 7.500 |
| Total (Core plus coating plus subcoating) | 7.310 | 10.965 |

TABLE 17
Coatings (amounts given in mg)

| Coating | e | f |
|---|---|---|
| Eudragit L 30 D aqueous dispersion (30%) - (dry) | 1.500 | 2.250 |
| Triethylcitrate | 0.300 | 0.450 |
| Colloidal silicon dioxide | 0.200 | 0.300 |
| Water | q.s. | q.s. |
| Subcoating | | |
| Hydroxypropylmethylcellulose 3 cps | 0.125 | 0.188 |
| Ethylcellulose 30% aqueous dispersion - (dry) | 0.125 | 0.188 |
| Triethylcitrate | 0.100 | 0.150 |
| Talc | 0.100 | 0.150 |
| Water | q.s. | q.s. |
| Total (coating plus subcoating) | 2.450 | 3.676 |
| Core | 5.000 | 7.500 |
| Total (Core plus coating plus subcoating) | 7.450 | 11.176 |

TABLE 18
Coatings (amounts given in mg)

| Coating | i | j |
|---|---|---|
| Eudragit L 30 D aqueous dispersion | 1.500 | 2.250 |
| (30%) - (dry) | | |
| Triethylcitrate | 0.300 | 0.450 |
| Talc | 0.200 | 0.300 |
| Water | q.s. | q.s. |
| Total (coating) | 2.000 | 3.000 |
| Core | 5.000 | 7.500 |
| Total (Core plus Coating) | 7.000 | 10.500 |

TABLE 19
Coatings (amounts given in mg)

| Coating | k | l |
|---|---|---|
| Hydroxypropylmethylcellulose | 2.000 | 3.000 |
| acetate succinate | | |
| Triethylcitrate | 0.600 | 0.900 |
| Talc | 0.400 | 0.600 |
| Water | q.s. | q.s. |
| Total (coating) | 3.000 | 4.500 |
| Core | 5.000 | 7.500 |
| Total (Core plus Coating) | 8.000 | 12.000 |

TABLE 20
Coatings (amounts given in mg)

| Coating | m | n |
|---|---|---|
| Eudragit L-100-55 | 1.500 | 2.250 |
| Triethylcitrate | 0.150 | 0.225 |
| Colloidal silicon dioxide | 0.500 | 0.750 |
| Isopropanol/water 97:3 | q.s. | q.s. |
| Total (coating) | 2.150 | 3.225 |
| Core | 5.000 | 7.500 |
| Total (Core plus Coating) | 7.150 | 10.725 |

TABLE 21
Coatings (amounts given in mg)

| Coating 10 | o | p |
|---|---|---|
| Eudragit L 30 D aqueous dispersion (30%) - dry | 1.500 | 2.250 |
| Triacetine | 0.150 | 0.225 |
| Glycerinmonostearate | 0.450 | 0.675 |
| Water | q.s. | q.s. |
| Total (coating) | 2.100 | 3.150 |
| Core | 5.000 | 7.500 |
| Total (Core plus Coating) | 7.100 | 10.650 |

The coated minitablets may be filled into hard gelatine capsules as defined in Example 1, e.g. 60 minitablets having the composition of Table 11 may be filled in a hard gelatine capsule of size 00, or 40 minitablets having the composition of Table 12 or 13 may be filled in a hard gelatine capsule of size 0.120 coated minitablets may also be filled in a sachet to give a dose of 720 mg MPA.

### EXAMPLE 10

1. Preparation of Drug Microparticles

A polymer solution is firstly prepared by dissolving the cellulose acetate phthalate and the polyethylene in cyclohexane with heating and stirring. Subsequently, the drug and the stabilizer are added and the dispersion allowed to cool whilst stirring. The resultant coated microparticles are washed and dried and then coated with one of the enteric coating formulations 1 or 2 below.

Composition (amounts given in %) of the Core

| | | | |
|---|---|---|---|
| MPA, Na Mycophenolate or MMF | 74% | 79% | 84% |
| Cellulose acetate phthalate | 21% | 16% | 11% |
| Polyethylene | 1% | 1% | 1% |
| Colloidal silica (Syloid ®) | 4% | 4% | 4% |
| Cyclohexane | qs* | qs* | qs* |

*removed during processing

Enteric coated drug microparticles may be formulated into a capsule or sachet by the addition of bulking agents and lubricants or further compressed into tablets or minitablets.

2. Preparation of Granules

A dry blend is made by mixing the drug, Aerosil 200, Povidone (PVP) K30 and lactose in a planetary or high shear mixer. Ethanol is added to produce granules which are thoroughly dried and sieved for suitable size selection. The resulting granules finally are coated with an aqueous solution of the enteric coating ingredients (coating 1 below) or with an organic solution of the enteric coating ingredients (coating 2 below).

Composition (amounts given in %) of the Core

| | | | |
|---|---|---|---|
| MPA, Na Mycophenolate or MMF | 50% | 30% | 60% |
| Povidone K-30 | 5% | 5% | 5% |
| Aerosil 200 | 2% | 2% | 2% |
| Ethanol 94% for granulation | qs | qs | qs |
| Lactose | 43% | 63% | 33% |

3. Preparation of Pellets:

A dry blend is made by mixing the drug, microcrystalline cellulose (Avicel PH101) and lactose in a planetary mixer. Purified water is added to give a wet mass that is subsequently extruded using a screen of a suitable size. The extrudates are rounded in a spheroniser, thoroughly dried and sieved for suitable size selection. The resulting pellets finally are coated with an aqueous solution of the enteric coating ingredients (coating 1 below) or with an organic solution of the enteric coating ingredients (coating 2 below).

Composition (amounts given in %) of the Core

| | | | |
|---|---|---|---|
| MPA, Na Mycophenolate or MMF | 50% | 30% | 60% |
| Lactose (standard grade) | 25% | 35% | 20% |
| Microcrystalline cellulose (Avicel PH1) | 25% | 35% | 20% |
| Water for wet massing | q.s.* | q.s.* | q.s.* |

*removed during processing.

4. Preparation of Beads

Drug solution are prepared by dissolving the drug, and the formulation components as described in formulations/table A & B in the selected media with mixing.

Formulation A

Non-pareil seeds are dispensed into a Wurster fluid bed coater and fluidized. The drug solution previously prepared is then sprayed onto the seeds until the drug solution is depleted. The beads are dried in the same conditions for 5 minutes. The beads of formulation A are then finally coated with an aqueous solution of the enteric coating ingredients (coating 1 below) or with an organic solution of the enteric coating ingredients (coating 2 below) and dried for 15 minutes. Optionally a subcoating as indicated in Table 17 can be applied. Beads can then be dispensed in a capsule or sachet.

Formulation B

Non-pareil seeds are dispensed into a Wurster fluid bed coater and fluidized. The drug solution previously prepared is then sprayed onto the seeds until the drug solution is depleted. The beads are then sprayed with a solution of hydroxypropy methylcellulose (Opadry) in water and finally dried for 10 minutes. Optionally a subcoating as indicated in Table 17 can be applied. Beads can then dispensed in a capsule or sachet.

Formulations to be Applied Onto 1000 g Non-Pareil Seeds: Composition (Amounts Given in %)

a) Formulation A

| | | | |
|---|---|---|---|
| MPA, Na Mycophenolate or MMF | 80% | 60% | 40% |
| Hydroxypropyl methylcellulose(Methocel E50LV) | 18% | 36% | 54% |
| Polyethylene glycol (PEG 400) | 2% | 4% | 6% |
| Ethanol/Water (70:30) | q.s.* | q.s.* | q.s.* |

*removed during processing.

b) Formulation B

| | | | |
|---|---|---|---|
| Compound A | 80% | 60% | 40% |
| Talc | 8% | 15% | 24% |
| Hydroxypropyl methylcellulose(Opadry) | 12% | 25% | 36% |
| Water | q.s.* | q.s.* | q.s.* |

*removed during processing.

Beads for formulations A and B can be used as a combination by including them into the same capsule or sachet.

Alternately, beads can also be prepared by combining formulations A and B onto the same non-pareil seeds according to the following process. Formulation A is firstly sprayed onto the beads, followed by the enteric coating and finally formulation B. Optionally a subcoating can also be applied as described above.

Coating Formulations

Coating 1: enteric coating
Composition (amounts given in %)

| | |
|---|---|
| Eudragit L 30 D (dry) | 75% |
| Triacetine | 17.5% |
| Syloid 244 FP | 7.5% |
| Water | qs |

Coating 2: enteric coating
Composition (amounts given in %)

| | |
|---|---|
| HP 50 (dry) | 72% |
| Triethylcitrate | 7% |
| Colloidal silicon dioxide | 21% |
| Acetone, Ethanol 94% 1:1 | qs |

The minitablets of Example 1 may also be coated with an aqueous solution of the coating ingredients 1 above or with an organic solution of the enteric coating ingredients 2 above.

In the above examples, Compound A may be replaced by rapamycin or another rapamycin derivative, and/or sodium mycophenolate may be replaced by mycophenolate mofetil.

### EXAMPLE 11

Enteric coated pellets are mixed with the other ingredients and compressed on a rotary tablet press into tablets (one 834 mg oblong tablet corresponds to 180 mg mycophenolic acid)

| Composition | % | mg |
|---|---|---|
| enteric coated pellets | 50% | 417.0 |
| Sodium mycophenolate | | 192.4 (60% of the pellet) |
| Pellet core excipients | | 128.4 |
| Pellet enteric coating | | 96.2 |
| MCC (Avicel pH 101) | 22% | 183.5 |
| Avicel granulate | 21% | 175.2 |
| Crospovidone | 6% | 50.0 |
| Magnesium stearate | 1% | 8.3 |
| Total | 100% | 834.0 |

The bioavailability characteristics of the compositions of the invention may be determined in vivo in conventional manner, e.g. in dogs. They are also ascertained in standard clinical bioavailability trials. For example the compositions of the Examples may be administered to 12 healthy volunteers in single doses in a cross-over trial. AUC and Cₘₐₓ are measured.

1. A composition comprising mycophenolic acid, a salt or a prodrug thereof in multiparticulate form.

2. A composition comprising mycophenolic acid, a salt or a prodrug thereof, adapted to disintegrate or dissolve in the mouth, stomach or small intestine to give multiparticles.

3. A composition according to claim 1 wherein the multiparticulate form or the multiparticles are microparticles, minitablets, pellets, granules or beads.

4. A composition according to claim 3 in the form of minitablets, wherein the mean total weight of the minitablets is 3 to 10 mg.

5. A composition according to claim 3 in the form of microparticles having an average size of less than 1000 μm.

6. A composition according to claim 3 in the form of granules, pellets or beads having a diameter of from 0.2 to 2 mm.

7. A composition according to claim 1, wherein the multiparticles are enteric coated.

8. A composition according to claim 1, comprising one or more excipients selected from a binder, a filler, a disintegrant and a lubricant.

9. A composition according to claim 1, wherein the enteric coating comprises 15 to 50% of the total weight of the multiparticles.

10. A composition according to claim 1, further comprising a subcoating.

11. A composition according to claim 10 wherein the subcoating comprises hydroxypropylmethylcellulose or ethylcellulose.

12. A composition according to claims 1 comprising mycophenolate mofetil or sodium mycophenolate.

13. A fixed combination comprising a) mycophenolic acid, a salt thereof or a prodrug thereof and b) rapamycin or a rapamycin derivative.

14. A combination according to claim 13 wherein the active substances a) and b) are formulated
separately in subunits which are then combined in a single administration unit; or
in a common administration unit.

15. A combination comprising a) mycophenolic acid, a salt thereof or a prodrug thereof and b) rapamycin or a rapamycin derivative, wherein the subunits or administration units comprising mycophenolic acid, a salt thereof or a prodrug thereof are in the form of a composition according to claim 1.

16. A combination according to claim 15 wherein the mycophenolic acid, a salt thereof or a prodrug thereof is formulated into minitablets, or the rapamycin or rapamycin derivative is formulated into a tablet or into minitablets.

17. A composition or a combination according to any preceding claim for use in the treatment or prevention of native or transgenic organ, tissue or cellular allograft or xenograft transplant rejection, or treatment or prevention of autoimmune diseases.

18. A method for reducing inter- and intrapatient variability in a subject comprising administering a therapeutically effective amount of a composition comprising as active ingredient mycophenolic acid, a salt thereof or a prodrug thereof, wherein the composition is in a particulate form.
